(19) United States
(12) Patent Application Publication
(10) Pub. No.: US 2007/0015845 A1
(43) Pub. Date: Jan. 18, 2007

(54) DENTAL RESIN COMPOSITION, METHOD OF MANUFACTURE, AND METHOD OF USE THEREOF

(75) Inventors: Shuhua Jin, Wallingford, CT (US); Weitao Jia, Wallingford, CT (US)

Correspondence Address: CANTOR COLBURN, LLP, 55 GRIFFIN ROAD SOUTH, BLOOMFIELD, CT, 06002, US

(73) Assignee: PENTRON CLINICAL TECHNOLOGIES, LLC, Wallingford, CT (US)

(21) Appl. No.: 11/456,117

(22) Filed: Jul. 7, 2006

Related U.S. Application Data
(60) Provisional application No. 60/699,751, filed on Jul. 15, 2005.

(51) Int. Cl. A61L 24/00

(52) U.S. Cl. 523/113

(57) ABSTRACT

A composition is disclosed comprising a polymerizable oxetane-(meth)acrylate of the structure I: 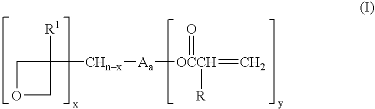
wherein R is hydrogen or methyl, R¹ is a C₁₋₆ alkyl group, n is 0-3, x is 1-3, y is 1-3, a is zero or one, and A is a linking group having the valency 1+y; and an effective amount of a cure initiator. The composition finds use as a dental resin.

### CROSS-REFERENCE TO RELATED APPLICATION

This application claims priority to U.S. Provisional Application Ser. No. 60/699,751 filed Jul. 15, 2005, which is incorporated by reference herein in its entirety.

### BACKGROUND

This invention relates to dental resin compositions comprising both cationic polymerizable oxetane and free radical polymerizable oxetane-(meth)acrylate resins, their method of manufacture, and the use of such resins for restorative dentistry, including dental adhesives, dental cements, dental filling materials, root canal sealants, crown and bridge materials, and the like.

In recent years, materials used for dental restorations have principally comprised acrylate or methacrylate resins. Resinous materials of this type are disclosed, for example, in U.S. Pat. No. 3,066,112 to Bowen, U.S. Pat. No. 3,194,784 to Bowen, and U.S. Pat. No. 3,926,906 to Lee et al. An especially important methacrylate monomer is the condensation product of bisphenol A and glycidyl methacrylate, 2,2′-bis [4-(3-methacryloxy-2-hydroxypropoxy)-phenyl]-propane (“BisGMA”). Alternatively, BisGMA can be synthesized from the diglycidyl ether of bisphenol A and methacrylic acid (see, e.g., U.S. Pat. No. 3,066,112 to Bowen).

Because the wear and abrasion characteristics and the overall physical, mechanical, and optical properties of these unfilled acrylic resinous materials is poor, and because acrylic resin systems exhibit high coefficients of thermal expansion relative to the coefficient of thermal expansion of the tooth structure, these substances by themselves are less than satisfactory. In particular, the disparity in thermal expansion coupled with high shrinkage upon polymerization results in poor marginal adaptability, and ultimately leads to secondary decay. Composite dental restorative materials containing acrylate or methacrylate resins and fillers were thus developed. The fillers are generally inorganic materials based on silica, silicate based glasses, or quartz. These filled compositions are useful for a variety of dental treatments and restorative functions including crown and bridge materials, fillings, adhesives, sealants, luting agents or cements, denture base materials, orthodontic materials and sealants, and other dental restorative materials.

Despite their suitability for their intended purposes, however, there is a perceived need in the art for improved polymerizable dental resin materials. New resins are therefore constantly being developed. For example, U.S. Patent Publication No. 2004/0242723 describes a new resin type that incorporates a methacrylate group and an epoxy group in the same molecule. Nonetheless, there remains a need in the art for dental resin materials that have improved properties, for example high strength, good biocompatibility, good bonding adhesion to a dental substrate, and/or minimal shrinkage upon polymerization without sacrificing other advantageous physical properties.

### SUMMARY

The above-described need in the art is met by a dental composition comprising a cationic-polymerizable oxetane and free radical-polymerizable oxetane-(meth)acrylate of general structure I: 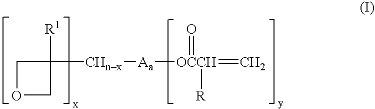
wherein R is hydrogen or methyl, R¹ is a C₁₋₆ alkyl group, n is 0-3, x is 1-3, y is 1-3, a is zero or one, and A is a linking group having the valency 1+y; and an effective amount of a cure initiator.

In another embodiment, a method of manufacturing a polymerizable dental composition comprises combining a polymerizable oxetane-(meth)acrylate of structure I with a cure initiator.

In yet another embodiment, a method of making a dental restoration comprises applying to a site to be restored a composition comprising the above-described polymerizable oxetane-(meth)acrylate of general structure I, and polymerizing the (meth)acrylate.

### DETAILED DESCRIPTION

The polymerizable oxetane (meth)acrylates described herein contain an oxetane group and a (meth)acrylate group. An oxetane is a four-membered cyclic ether compound. Similarly to epoxides, oxetanes are reactive in the presence of ultraviolet and visible light by a cationic reaction mechanism, although oxetanes may need higher energy for the ring-opening of four-membered ring than three-member epoxides. The initiation of ring-opening reaction of oxetane can be slower than epoxides, but when both epoxides and oxetanes are used together, the polymerization rate can be enhanced. Furthermore, the use of free radical polymerization can produce heat to further enhance the polymerization rate. Without being bound by theory, it is believed that the combination of free radical and cationic polymerization of the resins described herein can accordingly result in lower polymerization shrinkage compared to the free radical-only polymerization of (meth)acrylates, while maintaining or even improving the properties of the cured product. Thus, the resins are useful as dental resins and can possess improved properties over existing dental resins, and correspondingly enhance the properties of dental restorative materials prepared from such resins. For instance, the polymerizable oxetane-(meth)acrylates can provide excellent bonding strength between a dental substrate (dentin, enamel, or other tooth structure) and the dental restorative material made from the polymerizable (meth)acrylate.

In particular, an improved polymerizable oxetane-(meth)acrylate is of formula I: 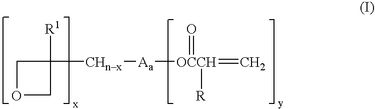
wherein R is hydrogen or methyl; R¹ is a an alkyl group having 1 to 6 carbon atoms; n is 0-3; x is 1-3; y is 1-3; a is 0 or 1; and A is a linking group having a valency of 1+y and 1 to about 100 carbon atoms. The linking group A can be unsubstituted or substituted, and is limited only to the extent that it is synthetically achievable, and does not significantly adversely affect the stability of the uncured resin or the properties of the cured resin.

In a specific embodiment, R¹ is methyl, ethyl, propyl, isopropyl, n-butyl, sec-butyl, or tert-butyl, a is 0 and R is hydrogen or methyl. In still another specific embodiment, R¹ is ethyl, a is 0 and R is methyl, thereby providing structure II.

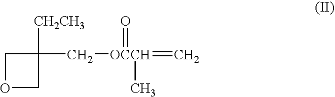

This oxetane, also known as 3-ethyl-3-oxetanyl)methoxymethylmethacrylate, is commercially available under trade name ETERNACOLL® OXMA from UBE America Inc.

When the subscript a in Formula I is 1, one suitable linking group is a polyether, for example a polyether of the formula —[OB]ₙOD-, wherein n is 1 to about 10, B is a substituted or unsubstituted C₁₋₃₂ alkylene, aralkylene, alkarylene, arylene, bis(alkylaryl), or bis(arylalkyl) group, and D is a substituted or unsubstituted C₁₋₁₂ alkylene group of the appropriate valency, e.g., 2. Such compounds have the structure shown in formula III: 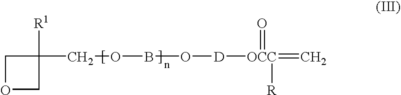
wherein n is an integer from 1 to 10, and R, R¹, and D are as defined above.

In one embodiment, B is an (bis arylenealkylene) group, including a substituted or unsubstituted bis(phenylalkyl) group wherein the alkyl groups have 1 to 4 carbon atoms. Specifically, B is a bis(phenylenemethylene) group. Such compounds are of structure IV.

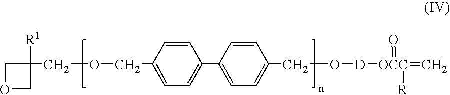

D can specifically be a substituted or unsubstituted, branched alkylene group having 5 to about 12 carbon atoms, specifically 6 to about 8 carbon atoms. In one embodiment, D is substituted with one or more hydroxyl groups.

A specific example of a compound of formula IV has the structure shown in formula V.

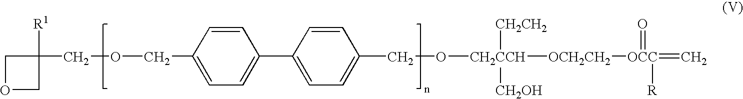

Another specific example of a compound of formula IV has the structure shown in formula VI:

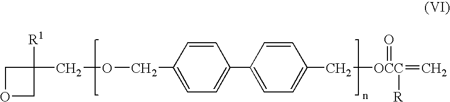

Oxetane (meth)acrylates of formula IV can be obtained by the reaction of a dioxetane and a hydroxy-containing (meth)acrylate. The reaction is conducted using more than one chemical equivalent of oxetane to hydroxyl group. Compounds of structure VI above can be obtained by reaction with acrylic acid or methacrylic acid, wherein the hydroxyl group is provided by the carboxylic acid. Compounds of structure V above can be obtained by reaction with a hydroxy-containing (meth)acrylate of the following formula: 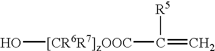
wherein R6 and R⁷ are each independently hydrogen, hydroxy, C₁-C₁₂ alkyl, C₁-C₁₂ perhaloalkyl, C₁-C₁₂ alkoxy, C₁-C₁₂ perhaloalkoxy, C₂-C₁₂ alkenyl, C₂-C₁₂ alkynyl, (C₁-C₆ alkyl)-O—(C₁-C₆ alkylene), or hydroxy(C₁-C₆ alkylene); z is an integer from 1 to 10; and R⁵ is hydrogen or methyl. In one embodiment, the hydroxy-containing (meth)acrylate is a hydroalkyl(meth)acrylate having 5 to 12 carbon atoms. For example, 4,4′-bis[3-ethyl-3-oxetanyl)methoxymethyl]biphenyl, having structure VI, 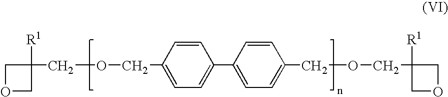
is commercially available under trade name ETERNACOLL® OXBP, also from UBE America Inc. This compound can be reacted with hydroxyethyl methacrylate (HEMA) to produce oxetane (meth)acrylate V. It can be seen that in this reaction, the alkylene group corresponding to D in formula IV is derived from one of the oxetane groups and the alkyl group of the hydroxyalkyl(meth)acrylate.

In one process, in one manner of proceeding, the di-oxetane and hydroxyl-group containing(meth)acrylate are mixed for a period of time at elevated temperature, for example from 120 to 250° C. The use of catalysts will accelerate the reaction. Suitable catalysts include a Lewis acid or a tertiary amine, for example tin(II) 2-ethylhexanoate, toluene sulfonic acid or benzenedimethylamine.

It is generally desirable to use the catalyst in an amount of about 0.10 to about 10 mole percent based on the total moles of the reactant mixture. Within this range it is generally desirable to utilize the catalyst in an amount about 1 to about 8, specifically about 2 to about 7, and more specifically about 3 to about 6 mole percent based on the total moles of the reactants.

Similarly, a method of making a compound of formula I comprises reacting an oxetane of formula VII with acrylic acid or methacrylic acid under the above-described conditions.

The polymerizable oxetane-(meth)acrylates can be used alone or in combination with other co-polymerizable, ethylenically unsaturated monomers and/or oligomers. This can also be combined with epoxy-methacrylate as described in US 2004/0242723 and/or other epoxide resins. For example, one or more other co-polymerizable, ethylenically unsaturated monomers and/oligomers containing carboxylic acid(s), phosphoric acid(s), sulfonic acid(s) or their anhydride(s) can be utilized in combination with the polymerizable (meth)acrylates of this invention. Mixtures comprising the polymerizable oxetane-(meth)acrylate and other components such as polymerization initiators, additives, and fillers can be prepared to form dental materials suitable for use as dental adhesives, dental cements, dental filling materials, root canal sealing/filling materials, and/or other dental restorative materials such as crown and bridge materials, provisional crown and bridge materials, and the like. It is generally desirable to use the polymerizable oxetane-(meth)acrylate in an amount of about 1 to about 99 weight percent based on the total weight of the dental restorative material. Within this range it is generally desirable to use the polymerizable oxetane-(meth)acrylate in an amount of about 10 to about 95 weight percent, specifically about 30 to about 90 weight percent, and most specifically about 50 to about 80 weight percent based on the total weight of the dental restorative material.

Known viscous resins can be used in combination with the polymerizable oxetane-(meth)acrylate to provide a dental restorative material. Non-limiting examples include polyurethane dimethacrylates (PUDMA), diurethane dimethacrylates (DUDMA), and/or the polycarbonate dimethacrylate (PCDMA) disclosed in U.S. Pat. Nos. 5,276,068 and 5,444,104 to Waknine, which is the condensation product of two parts of a hydroxyalkylmethacrylate and 1 part of a bis(chloroformate). Another advantageous resin having lower water sorption characteristics is an ethoxylated bisphenol A dimethacrylate (EBPDMA) as disclosed in U.S. Pat. No. 6,013,694 to Jia, et al. Still another useful resin material is disclosed in U.S. Pat. No. 6,787,629 to Jia, et al. An especially useful methacrylate resin is BisGMA.

Diluent monomers can be used to increase the surface wettability of the composition and/or to decrease the viscosity of the polymerization medium. Suitable diluent monomers include those known in the art such as hydroxyalkyl (meth)acrylates, for example 2-hydroxyethyl (meth)acrylate, 2-hydroxypropyl (meth)acrylate, and 4-hydroxybutyl (meth)acrylate; ethylene glycol (meth)acrylates, including ethylene glycol methacrylate, diethylene glycol methacrylate, tri(ethylene glycol) dimethacrylate and tetra(ethylene glycol) dimethacrylate; and diol dimethacrylates such as 1,4-butanediol di(meth)acrylate, dodecane diol di(meth)acrylate, or 1,6-hexanediol di(meth)acrylate, particularly 1,6-hexanediol dimethacrylate (HDDMA). Other suitable monomers include polyethylene glycol mono(meth)acrylate; glycerol di(meth)acrylate; trimethylolpropane di(meth)acrylate; pentaerythritol tri(meth)acrylate; the (meth)acrylate of phenyl glycidyl ether; and the like. Tri(ethylene glycol) dimethacrylate (TEGDMA) is particularly preferred.

Diluent monomers or viscous resins, when present, are incorporated into the dental restorative materials in an amount of about 1 to about 70 weight percent of the total dental restorative material.

The optional filler system can comprise one or more of the inorganic fillers currently used in dental composite materials. Preferred fillers include those, which are capable of being covalently bonded to the polymerizable oxetane-(meth)acrylate matrix itself or to a coupling agent (e.g., silanes) that is covalently bonded to both. Examples of suitable filling materials include but are not limited to, silica, quartz, strontium silicate, strontium borosilicate, lithium silicate, lithium alumina silicate, amorphous silica, ammoniated or deammoniated calcium phosphate, tricalcium phosphate alumina, zirconia, tin oxide, titania and combinations comprising at least one of the foregoing fillers. Some of the aforementioned inorganic filling materials and methods of preparation thereof are disclosed in U.S. Pat. No. 4,544,359 and U.S. Pat. No. 4,547,531 to Waknine, pertinent portions of which are incorporated herein by reference. Organic-inorganic fillers of POSS™ (Hybrid Plastics) can be incorporated into the composites as disclosed in U.S. Patent Application Publication 2002/0198282 A1. Other organic-inorganic fillers such as zirconium methacrylate and zirconium dimethacrylate under the codes of CXZR050 and CXZR051 (Gelest, Inc.) can also be used. Suitable high refractive index filler materials such as high refractive index silica glass fillers; calcium silicate based fillers such as apatites, hydroxyapatites or modified hydroxyapatite compositions can also be used. Alternatively, inert, non-toxic radiopaque materials such as bismuth oxide (Bi₂O₃), bismuth oxychloride, zirconium oxide, barium sulfate, and bismuth subcarbonate in micro- or nanoscaled sizes can be used. In addition, fibrous fillers such as those disclosed in U.S. Pat. Nos. 6,013,694, 6,403,676 and 6,270,562 to Jia and Jia et al. can also be used.

Suitable fillers have particle sizes of about 0.01 to about 5.0 micrometers, and can further comprise bound or unbound silicate colloids of about 0.001 to about 0.2 micrometers. These additional fillers can also be treated with a silane-coupling agent to increase adhesion with the polymerizable, (meth)acrylate. Commercially available silane treated fumed silica based on Aerosil A200 can be obtained from Degussa Corp under the names of Aerosil R711 and R7200.

The amount of total filler system in the dental restorative material can vary from about 1 to about 90 weight percent based on the total weight of the dental restorative material. The amount used is determined by the requirements of the particular application. Thus, for example, crown and bridge materials generally comprise about 60 to about 90 weight percent filler; luting cements comprise about 20 to about 80 weight percent filler; sealants generally comprise about 1 to about 20 weight percent filler; adhesives generally comprise about 1 to about 30 weight percent filler; and restorative materials comprise about 50 to about 90 weight percent filler, with the remainder in all cases being the polymerizable oxetane-(meth)acrylate and other optionally added resins.

The polymerizable oxetane-(meth)acrylate can be used together with a curing system, which generally includes polymerization initiators; polymerization accelerators; ultraviolet light absorbers; antioxidants; and other additives. In the instant case, because both (meth)acrylate and oxetane groups are present, the curing system can comprise a free radical-type initiator system and/or a cationic-type initiator system.

Suitable free radical polymerization initiators include initiators that can be utilized in UV-activated cure or visible light-activated cure compositions. For example, visible light-curable compositions employ light-sensitive compounds, including but not limited to benzil, benzoin, benzoin methyl ether, DL-camphorquinone (CQ), and benzil diketones. Either UV-activated cure or visible light-activated cure (approximately 230 to 750 nanometers) is acceptable. The amount of photoinitiator is selected according to the curing rate desired. A minimal catalytically effective amount is generally about 0.01 weight percent of the total dental resin composition, and will lead to a slower cure. Faster rates of cure are achieved with amounts of catalyst in the range from greater than about 0.01 weight percent to about 5 weight percent of the total dental resin composition. The total dental resin composition is the total weight of the polymerizable oxetane-(meth)acrylate and other resinous materials, such as for example, resinous diluents, which are used in the dental restorative material.

Alternatively, the free radical initiator can be formulated as a self-curing system. Self-curing dental composite materials will generally contain free radical polymerization initiators such as, for example, a peroxide in an amount of about 0.01 to about 1.0 weight percent of the total resin dental composite material. Particularly suitable free radical initiators are lauryl peroxide, tributyl hydroperoxide and, more particularly benzoyl peroxide (BPO).

Free radical-type polymerization accelerators suitable for use include the various organic tertiary amines well known in the art. In visible light-curable dental restorative materials, the tertiary amines are generally (meth)acrylate derivatives such as dimethylaminoethyl methacrylate and, particularly, diethylaminoethyl methacrylate (DEAEMA) or tertiary aromatic amines such as ethyl 4-(dimethylamino)benzoate (EDMAB) in an amount of about 0.05 to about 2.0 weight percent of the total dental restorative material. In the self-curing dental composite materials, the tertiary amines are generally aromatic tertiary amines, preferably tertiary aromatic amines such as ethyl 4-(dimethylamino)benzoate (EDMAB), 2-[4-(dimethylamino)phenyl]ethanol, N,N-dimethyl-p-toluidine (DMPT), and bis(hydroxyethyl)-p-toluidine (DHEPT). Such accelerators are generally present in an amount of about 0.5 to about 4.0 weight percent of the total dental restorative material.

It is furthermore preferred to employ an ultraviolet absorber in an amount of about 0.05 to about 5.0 weight percent of the total dental restorative material. Such UV absorbers are particularly desirable in the visible light-curable dental restorative materials in order to avoid discoloration of the resin from incident ultraviolet light. Suitable UV absorbers are the various benzophenones, particularly UV-5411 available from American Cyanamid Company.

The oxetane-(meth)acrylate resin cure system can also include a cationic polymerization system, or a combination of binary curing systems of free radical and cationic polymerization, as described, for example, in U.S. Pat. No. 6,084,004. Cationic polymerization is usually triggered by Lewis or Bronsted acids. The acids can be added to the cationically curable formulation directly, or produced by prior chemical and, in particular, photochemical reactions. A number of photoinitiators that dissociate under the action of light of the wavelength range of 215 to 400 nm to form Bronsted acids include, for example, diazonium compounds (e.g., U.S. Pat. No. 3,205,157), sulphonium compounds (e.g., U.S. Pat. No. 4,173,476) and iodonium compounds (e.g., U.S. Pat. Nos. 4,264,703 and 4,394,403). The foregoing compounds are initiated in the presence of UV light. The amount of photoinitiator is selected according to the curing rate desired. A minimal catalytically effective amount is generally about 0.01 weight percent of the total dental resin composition, and will lead to a slower cure. Faster rates of cure are achieved with amounts of catalyst in the range from greater than about 0.01 weight percent to about 8 weight percent of the total dental resin composition. In one embodiment, the curing system comprises 0.01 to 8 weight percent, specifically 0.1 to 5 weight percent, of a diaryliodonium compound or a mixture of diaryliodonium compounds, 0.01 to 8 weight percent, specifically 0.1 to 5 weight percent, of an alpha-dicarbonyl compound, and 0.001 to 5 weight percent, specifically 0.01 to 3 weight percent, of an aromatic amine, each based on the total weight of the resin composition.

In one embodiment, the polymerizable oxetane-(meth)acrylate is prepared by reacting an aromatic compound comprising anhydride and/or carboxylic acid functionality with a hydroxy-containing (meth)acrylate monomer in the presence of a catalyst. The resulting polymerizable oxetane-(meth)acrylate is then formulated into a dental restorative material by mixing with the filler system and the curing system. The dental restorative material is then applied to the tooth to be repaired, and cured.

Alternatively, the dental restorative material can be formulated as a two-part system, wherein the first part can comprise the polymerizable oxetane-(meth)acrylate and the filler system. The second part can comprise the curing system and optional diluent monomers. When necessary, the two parts are metered out and then mixed using a spatula. The cure may be initiated through the use of UV light or by raising the temperature of the mixture. The dental restorative material thus obtained is then placed in the tooth to be restored after the tooth is appropriately prepared. Methods for use of the above-described compositions are well known in the art.

The invention is further illustrated by the following non-limiting examples.

### EXAMPLES 1-10

### Cationic and Free Radical Light Curable Resin Containing OXMA and/or OXBP

The cationic and free radical curable example resin compositions Example 1 to Example 10 were prepared as described in Table 1. All the compositions contain both a cationic photoinitiator diaryliodonium hexafluoroantimonate (CD1012, Sartomer, Pa., 3%), a radical photoinitiator CQ (0.2%) and an amine accelerator EDMAB (0.5%).

TABLE 1
Cationic and free radical light curable
resins containing OXMA and/or OXBP

| Resin wt % | OXMA | OXBP | BAHEMA | EBPADMA |
|---|---|---|---|---|
| Example 1 | 20 | 80 | | |
| Example 2 | 20 | | 80 | |
| Example 3 | 20 | | | 80 |
| Example 4 | 50 | 50 | | |
| Example 5 | 50 | | | 50 |
| Example 6 | 20 | 40 | 40 | |
| Example 7 | 20 | 20 | 60 | |
| Example 8 | 20 | 40 | | 40 |
| Example 9 | 20 | 20 | | 60 |
| Example 10 | 20 | | 40 | 40 |

Modulus of Rupture (MOR) of the Examples 1 -10 was measured using an ATS machine as per ISO 4049. The samples were cured for 2 minutes in and outside the mold using CureLite™ Plus curing light (Pentron Corp.) and stored in water at 37° C. for 24 hours. Vicker's Microhardness (VH) was measured using Clark™ Hardness Tester (Clark Instrument Inc.). The samples were cured for 20 seconds using Avante™ curing light (Pentron Corp.) and stored in water at 37° C. for 24 hours. Both MOR and VH were shown in Table 2.

TABLE 2
Mechanical properties of various resin compositions

| Examples | MOR (Psi) | VH (Kg/mm²) |
|---|---|---|
| Example 1 | Not measurable | Not measurable |
| Example 2 | 3436(716) | 14.9 |
| Example 3 | 15788(182) | 19.7 |
| Example 4 | Not measurable | Not measurable |
| Example 5 | Not measurable | 15.4 |
| Example 6 | 17153(449) | 15.9 |
| Example 7 | 14520(2218) | 15.7 |
| Example 8 | 14723(1567) | 13.6 |
| Example 9 | 16526(777) | 18.2 |
| Example 10 | 14350(878) | 18.6 |

### EXAMPLES 11-12

### Free Radical Light Curable Resin Containing OXMA

The free radical curable example resin compositions Example 11 to Example 12 were prepared as described in Table 3. All the compositions contain only free radical photoinitiator CQ (0.2%) and an amine accelerator EDMAB (0.5%).

TABLE 3
Free radical light curable resins containing OXMA

| Resin wt % | OXMA | BAHEMA | EBPADMA |
|---|---|---|---|
| Example 11 | 20 | 40 | 40 |
| Example 12 | 20 | 60 | 20 |

MOR was tested using the same method as described in Example 1, and is shown in Table 4.

TABLE 4
Mechanical properties of various resin compositions

| Examples | MOR (Psi) |
|---|---|
| Example 11 | 14942(442) |
| Example 12 | 11201(247) |

A composite of OXMA/BAHEMA/EBPADMA 20/50/30 was prepared with a composition of 24% of resin, 76% of amorphous silica and glass filler. The MOR is 17024(1093) Psi. The mechanical property of this composite is comparable to regular methacrylate composites.

As used herein, the term “(meth)acrylate” is intended to encompass both acrylate and methacrylate groups. The endpoints of all ranges directed to the same component or property inclusive of the endpoint and independently combinable. In addition, all patents are incorporated by reference in their entirety.

Suitable groups that may be present on a “substituted” position include, for example, halogen; cyano; hydroxyl; nitro; azido; alkanoyl (such as a C₂-C₆ alkanoyl group such as acyl or the like); carboxamido; alkyl groups, typically having 1 to about 8 carbon atoms, or 1 to about 6 carbon atoms; cycloalkyl groups, alkenyl and alkynyl groups, including groups having one or more unsaturated linkages and from 2 to about 8, or 2 to about 6 carbon atoms; alkoxy groups, including those having one or more ether linkages, and typically having 1 to about 8, or 1 to about 6 carbon atoms; aryloxy groups such as phenoxy; alkylthio groups, including those having one or more thioether linkages and 1 to about 8 carbon atoms, or 1 to about 6 carbon atoms; alkylsulfinyl groups, including those having one or more sulfinyl linkages and typically having 1 to about 8 carbon atoms, or 1 to about 6 carbon atoms; alkylsulfonyl groups, including those having one or more sulfonyl linkages and typically having 1 to about 8 carbon atoms, or 1 to about 6 carbon atoms; aminoalkyl groups, including those having one or more nitrogen atoms and typically 1 to about 8, or 1 to about 6 carbon atoms; aryl groups having 6 or more carbons and one or more rings, e.g., phenyl, biphenyl, naphthyl, or the like, each ring being either substituted or unsubstituted; arylalkyl groups having 1 to 3 separate or fused rings and typically 6 to about 18 ring carbon atoms, e.g., benzyl; arylalkoxy groups having 1 to 3 separate or fused rings and 6 to about 18 ring carbon atoms, e.g. benzyloxy; or a saturated, unsaturated, or aromatic heterocyclic group having 1 to 3 separate or fused rings with 3 to about 8 members per ring and one or more nitrogen, sulfur, or oxygen atoms, e.g. coumarinyl, quinolinyl, isoquinolinyl, quinazolinyl, pyridyl, pyrazinyl, pyrimidinyl, furanyl, pyrrolyl, thienyl, thiazolyl, triazinyl, oxazolyl, isoxazolyl, imidazolyl, indolyl, benzofuranyl, benzothiazolyl, tetrahydrofuranyl, tetrahydropyranyl, piperidinyl,

1. A dental restorative composition comprising a polymerizable oxetane-(meth)acrylate of the structure I: 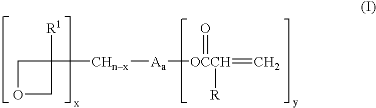
wherein R is hydrogen or methyl, R¹ is a C₁-₆ alkyl group, n is 0-3, x is 1-3, y is 1-3, a is or one, and A is a linking group having the valency 1+y; and
an effective amount of a cure initiator.

2. The composition of claim 1, wherein a is zero and R¹ is a C₁₋₄ alkyl

3. The composition of claim 2, wherein R is methyl and R¹ is ethyl.

4. The composition of claim 1, wherein a is 1, and A is a polyether.

5. The composition of claim 4, wherein the polyether is of the formula —[OB]ₙOD-, wherein n is 1 to about 10, B is a substituted or unsubstituted C₁₋₃₂ alkylene, aralkylene, alkarylene, arylene, bis(alkylaryl), or bis(arylalkyl) group, and D is a substituted or unsubstituted C₁₋₁₂ alkylene group.

6. The composition of claim 5, wherein B is a bis(arylenealkylene) group.

7. The composition of claim 5, having the structure of formula IV 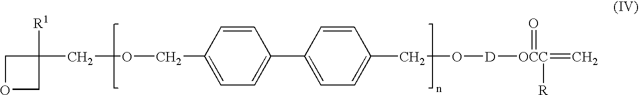
wherein D is a substituted or unsubstituted, branched alkylene group having 5 to about 12 carbon atoms.

8. The composition of claim 7, having the structure V:

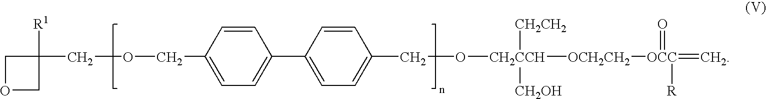

9. The composition of claim 7, having the structure VI:

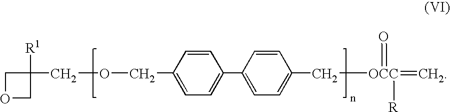

10. A method of making a compound of formula I, comprising reacting an oxetane of formula VII with a (meth)acrylic acid:

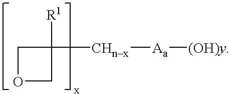

11. A method of making a compound of formula IV, comprising reacting a dioxetane having structure VI 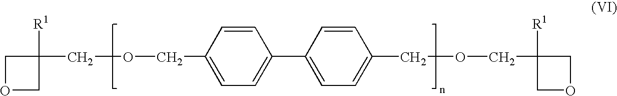
with acrylic acid, methacrylic acid, or a hydroxy-containing (meth)acrylate of the structure: 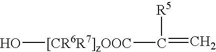
wherein R⁶ and R⁷ are each independently hydrogen, hydroxy, C₁-C₁₂ alkyl, C₁-C₁₂ perhaloalkyl, C₁-C₁₂ alkoxy, C₁-C₁₂ perhaloalkoxy, C₂-C₁₂ alkenyl, C₂-C₁₂ alkynyl, (C₁-C₆ alkyl)-O—(C₁-C₆ alkylene), or hydroxy(C₁-C₆ alkylene); z is an integer from 1 to 10; and R⁵ is hydrogen or methyl.

12. The composition of claim 1, comprising
about 1 to about 90 weight percent of a filler system based on the total weight of the composition.

13. The composition of claim 10, further comprising an additional ethylenically unsaturated monomer and/or oligomer that is co-curable with the polymerizable (meth)acrylate.

14. A method of making a dental restoration, comprising
applying to a site to be restored a composition comprising
a curing agent; and
a polymerizable oxetane-(meth)acrylate of claim 1; and
curing the composition to form a dental restoration.
